(19) United States
(12) Patent Application Publication
(10) Pub. No.: US 2018/0029326 A1
(43) Pub. Date: Feb. 1, 2018

(54) A COMPOSITE LAMINATE AND ITS USAGE

(71) Applicant: AGENCY FOR SCIENCE, TECHNOLOGY AND RESEARCH, Singapore (SG)

(72) Inventors: Yew Wei LEONG, Singapore (SG); Mohit SHARMA, Singapore (SG); Yongzheng PAN, Singapore (SG); Shilin CHEN, Singapore (SG)

(21) Appl. No.: 15/551,899

(22) PCT Filed: Feb. 17, 2016
(86) PCT No.: PCT/SG2016/050084

§ 371 Date: Aug. 17, 2017

(30) Foreign Application Priority Data
Feb. 17, 2015 (SG) 1020150123P

(51) Int. Cl. B32B 5/04; B29C 70/02; B32B 27/32; B32B 3/12; B32B 27/38

(52) U.S. Cl. CPC B32B 5/04; B32B 3/12; B32B 27/38; B32B 27/32; B29C 70/021; B32B 2305/74; B32B 2535/00; A61F 5/02

(57) ABSTRACT

The present invention relates to a laminate for making a molded article comprising: (i) at least one reinforcement Iayer impregnated with a resin matrix; (ii) at least one deployable layer; and (iii) optionally, at least one material comprising at least one non-adhesive side, wherein the deployable layer are compactable, expandable or collapsible including Miura-Ori folds, honeycombs, foams or air mesh. The laminate may be used to form a molded article. The molded articles have uses in biomedical, health care and sport protective devices.

### TECHNICAL FIELD

The present invention generally relates to laminates for making molded articles. The present invention also relates to the use of the molded articles made from the laminates which may be useful as biomedical, health care and sport protective devices.

### BACKGROUND ART

Laminates for molded articles are known and have many applications, such as in the fields of biomedical, health care and sport protective devices. Such molded articles have a use in the manufacturing of body braces for patients in need thereof.

Known laminates include those comprising an inner foam layer and an exterior shell. In such laminates, the material used for the shell part is polypropylene (PP). However, patients have complained that it is very uncomfortable since both the foam and PP shell are non-breathable. Therefore, there is a need for a molded article that provides suitable ventilation.

Furthermore, undulations between the foam and PP shell layers are common in those devices due to the low adhesion strength at the interface. In the manufacturing process of the molded articles, layers are normally laid separately onto the mold and then laminated via the application of heat and pressure, i.e. partial melting of the materials at the interface to form physical bonds. These bonds are weak, which causes the foam to delaminate from the shell after being worn for extended periods.

During manufacturing of conventional body braces or similar molded articles with a foam layer, the foam layer has to be initially wrapped around a polyurethane mold. Next, a sheet of PP is pre-heated in an oven to about 120° C. and softened to a pliable state and subsequently wrapped around the foam layer and formed as quickly as possible before the sheet becomes too cold and rigid. The assembly is then vacuum bagged to complete the forming process. This process involves many steps and requires skilled craftsmen to be able to form the brace or molded article accurately and effectively. Thus, the productivity in the manufacturing of such molded articles is not high.

Additionally, known laminates have sticky or tacky surfaces which makes it difficult for craftsmen to handle the material. In such laminates, due to the stickiness of the material, the external layer is lined with a non-permanent material, such as a plastic sheet, paper or outer polymer film liners, so as to allow for easier handling. This further contributes to the decrease in productivity in the manufacturing of molded articles and extra steps in its production due to the need to remove the non-permanent material.

Therefore, there is a need for a material that allows for an easier way of making and handling such molded articles which additionally shows an improved adhesion of the composite layers to the article. There is also a need to provide a material that can easily conform to different shapes while providing good ventilation without wrinkling or delamination between the composite layers. There is further a need for a material that provides good flexibility and ventilation while maintaining rigidity.

As such, there is a need to provide materials to make molded articles that overcome, or at least ameliorate, one or more of the disadvantages described above.

### SUMMARY OF INVENTION

In an aspect of the present disclosure, there is provided a laminate for making a molded article comprising:
(i) at least one reinforcement layer impregnated with a resin matrix;
(ii) at least one deployable layer; and
(iii) optionally, at least one material comprising at least one non-adhesive side.

Advantageously, the laminate is drapable and can be used easily to form a molded article with fewer process steps and without the need of special skills. For the manufacture of the molded article, the laminate may be fastened to the mold at room temperature, vacuum bagged and then left to cure at an elevated temperature to yield the appropriate shape. The resulting molded article may be thinner, lighter and much stronger than conventional articles, such as PP-based articles, due to the presence of the reinforcement in the laminate.

Further advantageously, the surface of the laminate may not be sticky or tacky which allows for easier handling and does not require the use of gloves.

Also advantageously, the resin may be used to bind the non-adhesive layer and the deployable layer to the reinforcement layer. Therefore, further adhesive layers would not be required. The laminate and/or resulting molded article may therefore be thinner and lighter than conventional articles, such as PP-based articles, or other articles which require the use of additional adhesive layer(s). Further advantageously, the adhesion of the non-adhesive layer and the deployable layer to the reinforcement layer is preserved even when the laminate is stretched during forming (i.e. without wrinkling or delamination between the layers).

Further advantageously, the deployable layer may be geometrically engineered to be deployable and/or collapsible which allows the laminate to easily conform to different shapes while providing good ventilation.

In another aspect, there is provided a process for making a laminate as defined above, comprising the following steps: providing a deployable layer; providing a reinforcement layer; impregnating the reinforcement layer with the resin matrix and partially curing the resin matrix; and contacting the reinforcement layer with the deployable layer and fully curing the resin matrix to form the laminate.

In another aspect, there is provided a process for making a laminate as defined above, comprising the following steps: providing a deployable layer; pre-impregnating a reinforcement fiber with a resin matrix and partially curing the resin matrix; weaving the reinforcement fiber to form a reinforcement layer; and contacting the reinforcement layer with the deployable layer and fully curing the resin matrix to form the laminate.

Advantageously, these steps can be performed with conventional equipment in a simple way.

In another aspect, there is provided a molded article that is obtainable by molding a laminate according to the present disclosure or by molding a laminate obtainable by any of the processes according to the present disclosure.

Advantageously, such molded articles are of lighter weight while retaining or improving on the stability of known molded articles. Therefore they can be used in applications where light weight and high stability are needed. For example, should the molded article be used as a light-weight body brace, it will reduce fatigue to the wearers of the brace who often need to walk long distances or exercise daily.

In addition, the molded article may possess improved ventilation when compared to known molded articles. This is advantageous in applications where this is desirable (e.g. braces in sport applications). Therefore, the present molded articles advantageously may not require holes to be drilled or punched into it. The resulting molded articles therefore possess improved structural integrity and mechanical strength.

In another aspect of the present disclosure, there is provided a method of use of the molded articles as a brace for scoliosis, a prosthetic, a sport protector or a safety device. Such devices possess improved mechanical strength, are light weight, have good ventilation, are easy to handle and are more resistant against problems of wear and tear.

### DEFINITIONS

The following words and terms used herein shall have the meaning indicated:

The term “laminate” as used herein refers to a composite material comprising at least two layers, for example, 2 layers, 3 layers, 4 layers, etc.

As used herein, the term “molding” refers to a process of manufacturing by shaping liquid or pliable raw materials, such as laminates, using a rigid or semi-rigid frame called a scaffold.

As used herein, the term “pre-preg” refers to “pre-impregnated” reinforcement fibers or fabrics where a matrix material, such as epoxy, is impregnated (infused) into the fibers or fabrics. The pre-preg may then be partially cured or B-staged so that the matrix becomes semi-solid and does not drip, thus providing ease of handling of the pre-preg material. Some pre-preg materials, especially those that cure at elevated temperatures, would require cold storage to prevent complete curing and prolong shelf life. There are other types of pre-pregs that do not need cold storage as their curing mechanisms are different, e.g. moisture-cured, IR light-cured.

As used herein, the term “non-adhesive” refers to a surface that generally has a low-affinity for binding or adhering to another surface.

As used herein, the term “deployable” refers to a material or layer that may be “folded-in” to make the material or layer more compact. The deployable material or layer may subsequently be “folded-out” to expand and occupy a larger area. Deployable structures have more degrees of freedom and therefore may be able to conform to different shapes and dimensions easily.

As used herein, the term “integral article” refers to a one-piece molded article. In an “integral article”, the layers comprising the article are permanently bonded to each other.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of the laminate will now be disclosed.

In a first aspect of the present disclosure, there is provided a laminate for making a molded article comprising:
(i) at least one reinforcement layer impregnated with a resin matrix;
(ii) at least one deployable layer; and
(iii) optionally, at least one material comprising at least one non-adhesive side.

The (iii) optionally at least one material comprising at least one non-adhesive side may be an external layer, the at least one reinforcement layer may be a middle layer, and the at least one deployable layer may be an inner layer. This means that when the laminate is used to form a molded article, the inner layer is the layer that is in contact with the surface of the mold, the external layer is the layer exposed to the environment and the middle layer lies between the inner and external layers.

In the disclosed laminate, the (ii) at least one deployable layer and (iii) optionally, at least one material comprising at least one non-adhesive side, may be adhered to the surface of the reinforcement layer(s) by the resin matrix. This advantageously means that the laminate may not comprise additional adhesive layer(s) to adhere the layers of the laminate to each other.

The resin matrix may be adhered to the at least one material comprising at least one non-adhesive side and deployable layer during the curing action of resin. The resin may flow during curing and penetrate partially to the at least one material comprising at least one non-adhesive side, which may cause adhesion. The protruded design on the deployable layer, may also facilitate the interlocking of the deployable layer to the reinforcement layer.

### Material Comprising at Least One Non-Adhesive Side

The material comprising at least one non-adhesive side may be optional.

The material may be chosen from a stretchable and breathable material. The material may be made from polyester, nylon, fibrous glass or glass fiber, carbon, aramid, polyolefin fabric, or combinations thereof. The material may be braided, woven, knitted, or combinations thereof.

The material may consist or comprise of an air mesh.

The air mesh may be a 3-dimensional warp-knitted fabric comprising yarns made of a material selected from the group consisting of nylon, polypropylene, polyester and any mixture thereof. The fabric structure may provide open spaces between the yarns and therefore facilitate breathability as well as provide stretchability.

The air mesh may be a permanent layer of the laminate. The air mesh may form the outer or external layer of the final product and may not be removed from the final product. The air mesh may not be removed prior to curing of the material. The air mesh may not be a sacrificial layer that does not form part of the final product. Consequently, the laminated comprising the air mesh may be an integral article.

The air mesh may provide better handling, breathability and a surface that is pleasant to touch. The air mesh may provide more functionality to the product and may also significantly reduce manufacturing time. This may be due to the air mesh comprising at least one non-adhesive side. The non-adhesive side may not be the side that is exposed to the environment. As the non-adhesive side is not sticky or tacky, this may provide for better handling of the laminate.

When the material consists or comprises a knitted structure, a variety of knitting techniques for the material can be chosen in consideration of the end product design, desired weight, conformability etc. Preferably, the knitting structure should be fine enough so that the resin from the reinforcement layer does not leach out to the outer surface of the laminate during B-stage and/or curing.

The material may comprise at least one non-adhesive side. The non-adhesive side may be an exposed side, i.e. the non-adhesive side is the side that is not adhered to the other layers of the laminate. This advantageously means that the surface of the laminate or subsequently molded article may not be sticky or tacky which allows for easier handling and may not require the use of gloves.

The material comprising at least one non-adhesive side may be a permanent layer, i.e. the material would not have to be removed after forming the laminate or subsequently formed molded article. The material comprising at least one non-adhesive side may be permanently bonded to the reinforcement layer. The material may be permanently bonded to the reinforcement layer by oven curing the laminate such that the laminate comprising the material comprising at least one non-adhesive side, the reinforcement layer and the deployable layer are an integral article. A subsequently formed molded article comprising the material comprising at least one non-adhesive side, the reinforcement layer and the deployable layer may also be an integral article. This advantageously means that the process for forming the laminate or subsequently formed molded article comprises less steps and may therefore lead to improved productivity in manufacturing.

The material may have a thickness in the range of about 0.5 mm to about 250 mm, about 0.5 mm to about 1 mm, about 0.5 mm to about 2 mm, about 0.5 mm to about 5 mm, about 0.5 mm to about 10 mm, about 0.5 mm to about 20 mm, about 0.5 mm to about 50 mm, about 0.5 mm to about 100 mm, about 0.5 mm to about 200 mm, about 1 mm to about 2 mm, about 1 mm to about 5 mm, about 1 mm to about 10 mm, about 1 mm to about 20 mm, about 1 mm to about 50 mm, about 1 mm to about 100 mm, about 1 mm to about 200 mm, about 1 mm to about 250 mm, about 2 mm to about 5 mm, about 2 mm to about 10 mm, about 2 mm to about 20 mm, about 2 mm to about 50 mm, about 2 mm to about 100 mm, about 2 mm to about 200 mm, about 2 mm to about 250 mm, about 5 mm to about 10 mm, about 5 mm to about 20 mm, about 5 mm to about 50 mm, about 5 mm to about 100 mm, about 5 mm to about 200 mm, about 5 mm to about 250 mm, about 10 mm to about 20 mm, about 10 mm to about 50 mm, about 10 mm to about 100 mm, about 10 mm to about 200 mm, about 10 mm to about 250 mm, about 50 mm to about 100 mm, about 50 mm to about 200 mm, about 50 mm to about 250 mm, about 100 mm to about 200 mm, about 100 mm to about 250 mm, about 200 mm to about 250 mm, or any range or value falling within the range of 0.5 mm to 250mm.

The material layer may comprise one layer or multiple layers. The multiple layers may contain or comprise two to ten layers, two to four layers, two to six layers, two to eight layers, four to six layers, four to eight layers, four to ten layers, six to eight layers, six to ten layers or eight to ten layers. The material layer may comprise or consist of one layer, two layers, three layers, four layers, five layers, six layers, seven layers, eight layers, nine layers, or ten layers. Advantageously, the material layer may be used for cushioning.

Further advantageously, the material layer may be breathable, facilitating ventilation within the laminate.

### Reinforcement Layer

The material of the reinforcement layer may be chosen from typical solid materials that have a suitable mechanical strength and are stretchable. The material of the reinforcement layer may be a fiber material. The fiber material may be a carbon fiber, glass fiber, para-aramid fiber (such as Kevlar® or Twaron®), polymer fiber, or combinations thereof. The material of the reinforcement layer may be in the form of braided fabric, woven fabric, knitted fabric, roving strands, chopped strands, or combinations thereof. Preferably, the material of the reinforcement layer may be carbon fiber of glass fiber.

The reinforcement layer may be impregnated with a resin matrix. The reinforcement layer may be pre-impregnated with a resin matrix. For example, a reinforcement fiber of the reinforcement layer may be pre-impregnated with a resin matrix. The resin matrix may be widely selected from typical polymer resin matrixes that are used for the reinforcement of the fiber materials mentioned above. The polymer in the resin matrix may optionally be used together with suitable solvents, accelerators and/or cross-linking agents. The polymer matrix may additionally comprise a filler. The polymer resins of the resin matrix may be a thermosetting polymer resin, such as epoxy, polyester, unsaturated polyester, vinylester, acrylic, polyurethane, or thermoplastic polymer resins. The epoxy resin may be a low molecular weight polymer or higher molecular weight polymer which normally contain at least two epoxide groups. The epoxy resins may be bis-phenol A epoxy resin, bis-phenol F epoxy resin, aliphatic epoxy resin, such as glycidyl epoxy resins and cycloaliphatic epoxides, glycidylamine epoxy resin, or combinations thereof. The epoxy resin may be used together with an accelerator and/or a cross-linking agent and optionally may include a filler. The cross linking agent may be an amine.

Prior to resin impregnation of the reinforcement layer or reinforcement fiber(s), a filler may be added to the resin matrix. The filler may be added to achieve desirable capabilities of the molded article made from the laminate. For instance, a filler may be added to modify the viscosity of the resin to obtain extra flexibility and stretchability, a thermal conductive filler may be used to enhance the dispersion of the body heat of a wearer of a molded article on the body, a light reflecting particle can be added for increasing traffic safety of the wearer, or a structural rigidity increasing filler can be added for sport equipment.

The filler may be a nanofiller. Suitable fillers may include carbon nanotubes, silica, layered silicates, polyhedral oligomeric silsequioxanes, graphene oxide, or combinations thereof.

The filler may comprise about 0.5 wt % to about 25 wt % of the polymer matrix, about 0.5 wt % to about 1 wt % of the polymer matrix, about 0.5 wt % to about 2 wt % of the polymer matrix, about 0.5 wt % to about 5 wt % of the polymer matrix, about 0.5 wt % to about 10 wt % of the polymer matrix, about 0.5 wt % to about 20 wt % of the polymer matrix, about 1 wt % to about 2 wt % of the polymer matrix, about 1 wt % to about 5 wt % of the polymer matrix, about 1 wt % to about 10 wt % of the polymer matrix, about 1 wt % to about 20 wt % of the polymer matrix, about 1 wt % to about 25 wt % of the polymer matrix, about 2 wt % to about 5 wt % of the polymer matrix, about 2 wt % to about 10 wt % of the polymer matrix, about 2 wt % to about 20 wt % of the polymer matrix, about 2 wt % to about 10 wt % of the polymer matrix, about 2 wt % to about 20 wt % of the polymer matrix, about 2 wt % to about 25 wt % of the polymer matrix, about 5 wt % to about 10 wt % of the polymer matrix, about 5 wt % to about 20 wt % of the polymer matrix, about 5 wt % to about 25 wt % of the polymer matrix, about 10 wt % to about 20 wt % of the polymer matrix, about 10 wt % to about 25 wt % of the polymer matrix, about 20 wt % to about 25 wt % of the polymer matrix, or any range or value falling within 0.5 wt % to 5 wt %.

These fillers may advantageously modify the viscosity of the resin to obtain extra flexibility and stretchability, even after B-staging.

The resin may be impregnated into the reinforcement material by pressure, for example, with the use of a roller to ensure homogenous distribution of the resin through the reinforcement material. The resin matrix may also advantageously bind the non-adhesive layer and deployable layer to the reinforcement layer, thereby omitting the need for additional adhesive layers in the laminate. Additionally, the use of the resin layer to bind the non-adhesive layer and deployable layer may advantageously allow the preservation of the adhesion between reinforcement layer, deployable layer and the optional material comprising at least one non-adhesive side, even when the laminate is stretched during formation or when used to make a molded article (i.e. without wrinkling or delamination). Further advantageously, the disclosed laminate may be able to stretch and conform to 3-dimensional shapes without wrinkling or delamination.

The reinforcement layer may a thickness in the range of 0.01 mm to about 5 mm, about 0.01 mm to about 0.02 mm, about 0.01 mm to about 0.05 mm, about 0.01 mm to about 0.1 mm, about 0.01 mm to about 0.2 mm, about 0.01 mm to about 0.5 mm, about 0.01 mm to about 1 mm, about 0.01 mm to about 2 mm, about 0.02 mm to about 0.05 mm, about 0.02 mm to about 0.1 mm, about 0.02 mm to about 0.2 mm, about 0.02 mm to about 0.5 mm, about 0.02 mm to about 1 mm, about 0.02 mm to about 2 mm, about 0.02 mm to about 5 mm, about 0.05 mm to about 0.1 mm, about 0.05 mm to about 0.2 mm, about 0.05 mm to about 0.5 mm, about 0.05 mm to about 1 mm, about 0.05 mm to about 2 mm, about 0.05 mm to about 5 mm, about 0.1 mm to about 0.2 mm, about 0.1 mm to about 0.5 mm, about 0.1 mm to about 1 mm, about 0.1 mm to about 2 mm, about 0.1 mm to about 5 mm, about 0.2 mm to about 0.5 mm, about 0.2 mm to about 1 mm, about 0.2 mm to about 2 mm, about 0.2 mm to about 5 mm, about 0.2 mm to about 1 mm, about 0.2 mm to about 2 mm, about 0.2 mm to about 5 mm, about 0.5 mm to about 1 mm, about 0.5 mm to about 2 mm, about 0.5 mm to about 5 mm, about 1 mm to about 2 mm, about 1 mm to about 5 mm, about 2 mm to about 5 mm, or any range or value falling within 0.01 mm to 5 mm.

The reinforcement layer may comprise one layer or multiple layers of reinforcement material impregnated with the resin matrix. The weight and thickness of the reinforcement layer may be selected to tailor the properties of the reinforcement layer for the required application. When the reinforcement layer comprises multiple layers, an even higher strength and stability may be obtained in the resulting molded article.

The multiple layers of reinforcement material impregnated with the resin matrix may contain or comprise 2 to 50 layers, 2 to 5 layers, 2 to 10 layers, 2 to 20 layers, 5 to 10 layers, 5 to 20 layers, 5 to 50 layers, 10 to 20 layers, 10 to 50 layers, 20 to 50 layers, or any range or value falling within 2 to 50 layers.

### Deployable Layer

The material of the deployable layer may be chosen from a suitable flexible material. The material of the deployable layer may be a flexible polymeric material. The polymeric material may be in liquid or solidified form, and formed from polymers. Examples of such polymeric materials include elastomers, polyethylene or polyolefin, ethylene vinyl (EVA) acetate, neoprene, silicone rubber, polyurethane, polyvinylchloride, polystyrene, and polyimide. The polymeric material may be in the form of a foam. Polymeric foams with a closed cell structure may be used, such as closed cell polyethylene foams, (EVA) ethylene vinyl acetate foams or neoprene foams. Closed-cell foams do not have interconnected pores. Advantageously, closed-cell foams may provide improvements with regard to stability, low moisture absorption, and mechanical strength. The material of the deployable layer may be selected from breathable materials which may advantageously enhance the ventilation of the resulting molded article formed from the laminate.

The deployable layer comprises a deployable/collapsible structure which allows the deployable layer to easily conform to different shapes while providing good ventilation. To achieve a deployable layer, the deployable layer may comprise a pattern of folds thereby allowing the layer to be collapsed. The pattern of folds may comprise a grid of parallelograms, such as the Miura-Ori fold. The Miura-Ori fold, named after its inventor Professor Koryo Miura from Tokyo University, comprises a grid of parallelograms which allows for a sheet of material to be compacted down in two dimensions. The crease patterns of the Miura fold form a tessellation of the surface by parallelograms. Further, the use of Miura-Ori folds may allow in-plane airflow through its open channels. An example of the Miura-Ori fold is shown in FIG. 1.

Apart from the Miura-Ori, other deployable architecture such as honeycomb, stretchable foams or air-mesh may be used as long as the structure does not wrinkle when the structure is stretched or compressed. Stretchable foams and air-mesh may or may not have open cavities to allow in-plane airflow.

The deployable structure may have more degrees of freedom compared to rigid structures such as foam and therefore may be stretched and bent without wrinkling. This may allow the stretchable pre-peg to be advantageously wrapped around the deployable structure and conformed to any shape without wrinkling or buckling.

Advantageously, the deployable layer itself may allow the laminate to easily conform to the shape of any mold. The deployable layer may be stretched or collapsed to provide stretchability and good ventilation while maintaining the rigidity of the structure. Due to this stretchability, intricate shapes may be formed from the resulting molded article even without the use of vacuum bagging. Advantageously, even without vacuum bagging, the final product may be wrinkle-free.

Further advantageously, the deployable layer may provide good ventilation such that additional holes do not have to be punched or drilled into the laminate or resulting molded article which may disadvantageously be tedious and compromise on the structural integrity of the resulting molded article. A compromise in structural integrity may result in reduced strength of the article due to stress concentration points. In addition, it may not be easy to punch consistent holes into the fabric especially when the fabric is thick and made of strong reinforcement materials such as glass fiber. Advantageously, the Miura-Ori may inherently allow in-plane airflow through its open channels, facilitating better ventilation without compromising structural integrity.

The smaller angle of a parallelogram in the Miura-Ori fold may be in the range of about 60° to about 90°, about 62° to about 90°, about 64° to about 90°, about 66° to about 90°, about 68° to about 90°, about 70° to about 90°, about 72° to about 90°, about 74° to about 90°, about 76° to about 90°, about 78° to about 90°, about 80° to about 90°, about 82° to about 90°, about 84° to about 90°, about 86° to about 90°, about 88° to about 90°, or any range or value falling with 60° to 90°.

The larger angle of the parallelogram in the Miura-Ori fold may be about 90° to about 120°, about 92° to about 120°, about 94° to about 120°, about 96° to about 120°, about 98° to about 120°, about 100° to about 120°, about 102° to about 120°, about 104° to about 120°, about 106° to about 120°, about 108° to about 120°, about 110° to about 120°, about 112° to about 120°, about 114° to about 120°, about 116° to about 120°, about 118° to about 120°, or any range or value falling within 90° to about 120°.

For example, the parallelogram in the Miura-Ori fold may have about 60° and about 120° angles, about 60° and about 120° angles, about 62° and about 118° angles, about 64° and about 116° angles, about 66° and about 114° angles, about 68° and about 112° angles, about 70° and about 110° angles, about 72° and about 108° angles, about 74° and about 106° angles, about 76° and about 104° angles, about 78° and about 102° angles, about 80° and about 100° angles, about 82° and about 98° angles, about 84° and about 96° angles, about 86° and about 94° angles, about 88° and about 92° angles, or about 90° and about 90° angles.

The deployable layer may have a thickness in the range of about 0.2 mm to about 10 mm, about 0.2 mm to about 0.5 mm, about 0.2 mm to about 1 mm, about 0.2 mm to about 2 mm, about 0.2 mm to about 5 mm, about 0.5 mm to about 1 mm, about 0.5 mm to about 2 mm, about 0.5 mm to about 5 mm, about 0.5 mm to about 10 mm, about 1 mm to about 2 mm, about 1 mm to about 5 mm, about 1 mm to about 10 mm, about 2 mm to about 5mm, about 2 mm to about 10 mm, about 5 mm to about 10 mm, or any range or value falling within 0.2 mm to 10 mm

### Protrusions

The laminate may further comprising at least one anchoring protrusion.

The protrusion may provide an interlocking mechanism between the deployable Miura-Ori layer and the adjacent reinforcement layer. The protrusion may be in the form of a “mushroom-shaped” protrusion. The “mushroom-shaped” protrusion cap may penetrate through the reinforcement material and lock-in to provide adequate anchoring and adhesion between the layers. The number and size of protrusion anchoring points may depend on the density of the weaved structure of the reinforcement layer. A high density knit structure may require smaller but fewer anchoring points and vice versa.

### Process for Making Laminate

There is provided a process for making the laminate which comprises the following steps: providing a deployable layer; providing a reinforcement layer; impregnating the reinforcement layer with the resin matrix and partially curing the resin matrix; and contacting the reinforcement layer with the deployable layer and fully curing the resin matrix to form the laminate.

A process for making a laminate as defined above, may comprise the following steps: providing a deployable layer; pre-impregnating a reinforcement fiber with a resin matrix and partially curing the resin matrix; weaving the reinforcement fiber to form a reinforcement layer; and contacting the reinforcement layer with the deployable layer and fully curing the resin matrix to form the laminate.

The reinforcement fibers may or may not be pre-impregnated with the resin matrix. If the reinforcement fibers are pre-impregnated with the resin matrix, the resin matrix should be non-tacky so that they may be weaved easily. If the reinforcement fibers are not pre-impregnated, then the weaved reinforcement layer may be subsequently impregnated with the resin matrix.

The weaving may provide stretchability to the fabric since the reinforcement fibers are not stretchable. This stretchability may be important to allow the material to easily conform to shapes without the need of external holding forces, such as the use of molds or vacuum bags.

Reinforcement fibers/layers may be impregnated with the matrix resin to form a pre-preg. The pre-preg may then be partially cured. The partial curing may be B-staging. The pre-preg may be B-staged at a moderately elevated temperature in order to allow the resin to become semi-solid. The B-stage may be performed at a temperature in the range of about 30° C. to about 90° C., about 30° C. to about 50° C., about 30° C. to about 70° C., about 50° C. to about 70° C., about 50° C. to about 90° C., or about 70° C. to about 90° C. The B-stage of the reinforcement fiber may be performed after the reinforcement fibers have been impregnated with the resin matrix and before weaving the fibers. The B-stage of the reinforcement layer may be performed after the reinforcement layer has been impregnated with the resin matrix and before contacting with the deployable layer.

The reinforcement fiber may be selected from the group consisting of glass fiber, carbon fiber, polymeric fiber and any mixture thereof.

The reinforcement layer may be contacted with the deployment layer to form a multilayer assembly before fully curing the resin matrix.

The process may comprise the step of wrapping the multi-layer assembly around a scaffold before fully curing the resin matrix.

The scaffold may be any article that has a shape which the laminate will be shaped after.

The scaffold may be made from any lightweight and easily formable material. The scaffold may be made from a material selected from the group consisting of wax, polystyrene foam, plaster, clay and any mixture thereof.

The process may comprise the step of removing the scaffold after curing the resin matrix.

The scaffold material may or may not be removed depending on the manufacturing requirements and desired weight of the final product.

The full curing of the resin matrix may be performed at an elevated temperature. The full curing of the resin matrix may be performed at a temperature in the range of about 90° C. to about 180° C., about 90° C. to about 120° C., about 90° C. to about 150° C., about 120° C. to about 150° C., about 120° C. to about 180° C., or about 150° C. to about 180° C.

The full curing may cure the laminate so that the shape is set permanently. Once the laminate is fully cured, it may be very rigid and non-pliable.

The process may comprise the step of contacting the cured laminate with at least one material comprising at least one non-adhesive side.

Depending on the formulation of the epoxy resin, the pre-preg may be tacky or non-tacky. The tacky formulation may be used when other layers such as the air-mesh and Miura-Ori layers need to be adhered to the reinforcement pre-preg. A non-tacky version may be used if the material needs to be handled conveniently and the desired product requires only the rigid reinforcement as the outer-layer.

The disclosed laminate may be in the form of a sheet or a sock. Advantageously, the laminate may easily conform to 3-dimensional open-ended shapes by wrapping the material over a mandrel and subsequent thermal curing or photocatalytic reaction. This allows the fabrication of 3-dimensional structures without using complicated and/or expensive tools.

The process may comprise the step of anchoring the deployment layer to the reinforcement layer.

The anchoring may be done using an anchoring protrusion.

In another aspect of the present disclosure, there is provided a molded article that is obtainable by molding a laminate according to the invention or obtainable by any of the processes according the invention.

The molded article may have a structure comprising or consisting of the following layers:

(i) at least one reinforcement layer impregnated with a resin matrix;

(ii) at least one deployable layer; and

(iii) optionally, at least one material comprising at least one non-adhesive side.

In a fifth aspect, there is provided the corresponding method of using the molded article as a brace for scoliosis, a prosthetic, a sport protector or a safety device.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows an example of a Miura-Ori fold of the deployable layer in one embodiment of the present invention.

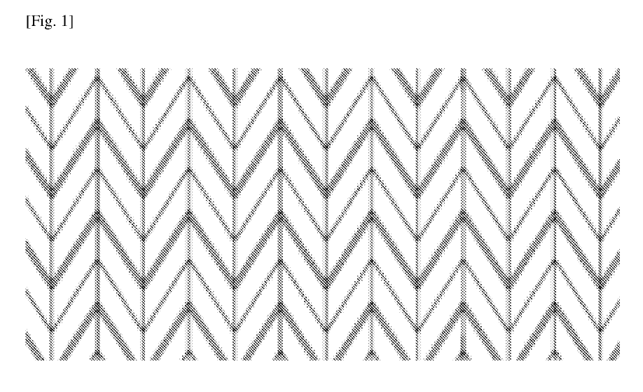

FIG. 2 shows a schematic representation of the preparation steps to produce a composite laminate according to one embodiment of the present invention.

FIG. 3 is an isometric view of the deployable layer showing breathable holes on the Miura-Ori folds on its top surface and anchoring protrusions on its bottom surface.

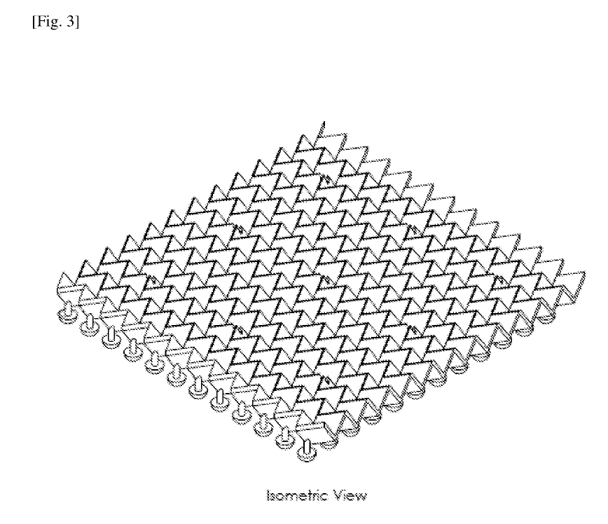

FIG. 4 shows a representation of the top view of the deployable layer comprising Miura-Ori folds, showing the size of each Miura-Ori fold, the size of each breathable hole, the positions of the breathable holes on the Miura-Ori folds and position of each anchoring protrusion. Dimensions shown in the figure are in millimeters.

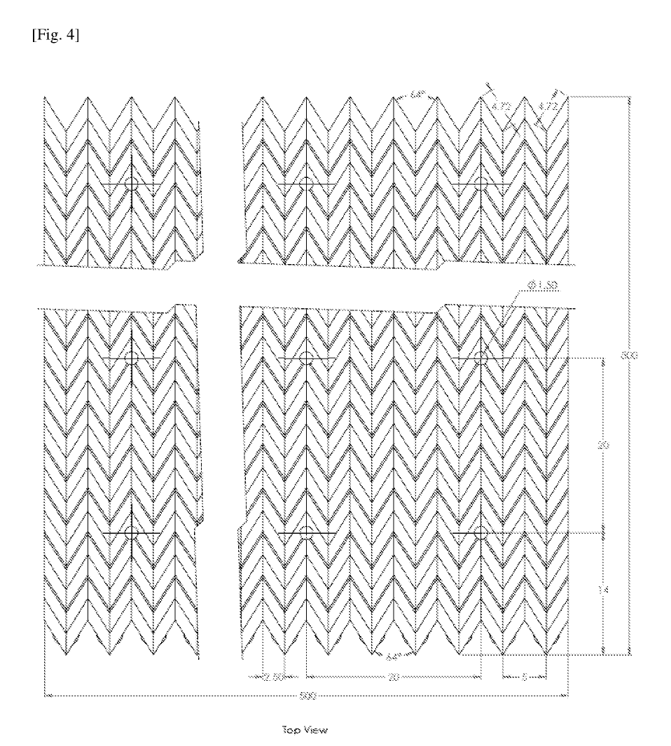

FIG. 5a shows a right-view representation of a deployable layer showing the size, shape and thickness of each Miura-Ori fold. Dimensions shown in the figure are in millimeters.

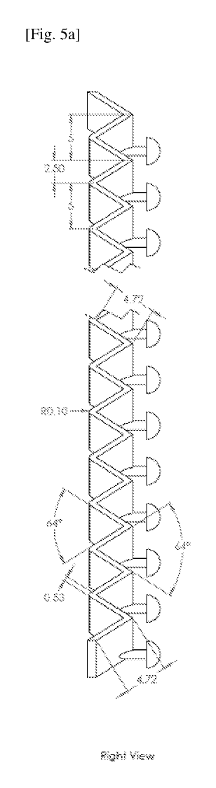

FIG. 5b shows a front-view representation of a deployable layer showing the thickness of the deployable layer, and the size and shape of each anchoring protrusion. Dimensions shown in the figure are in millimeters.

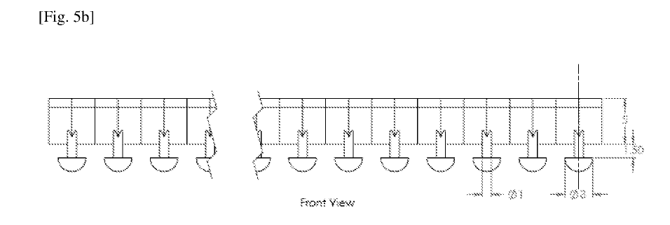

### DETAILED DESCRIPTION OF DRAWINGS

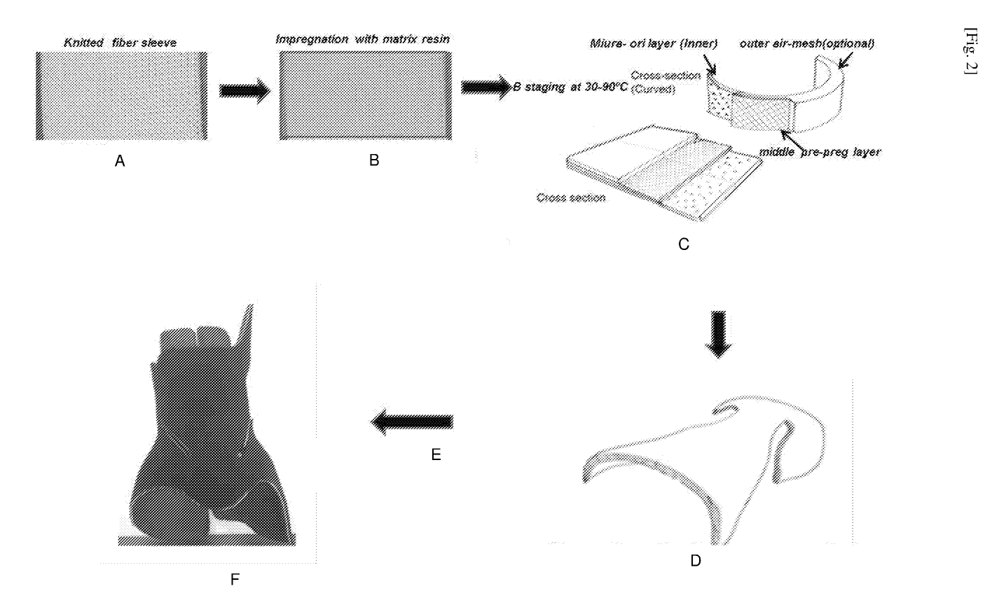

FIG. 2 shows a schematic representation of the preparation steps to produce a laminate of the present invention.

Step A: Reinforcement

The reinforcement fibers (glass fiber/carbon fiber/polymeric fiber) are knitted into the form of a sleeve or sock. The reinforcement fibers may or may not be pre-impregnated with the matrix resin. If the reinforcement fibers have been pre-impregnated with resin, the resin should be non-tacky so that they can be knitted easily. The knitting provides stretchability to the fabric since the reinforcement fibers are not stretchable. This stretchability may be important to allow the material to easily and snugly conform to shapes without the need of external holding forces, such as the use of molds or vacuum bags.

Step B: Impregnation

Reinforcements are impregnated with the matrix resin to form a pre-preg. The pre-preg is then B-staged at temperatures of between 30-90° C. in order to allow the resin to become semi-solid. This step is not necessary if the reinforcement fibers have been pre-impregnated with the matrix and B-staged prior to knitting the fibers into a fabric.

Step C: Wet Pre-Preg Wrapping Over Scaffold

Inner Miura-Ori layer is adhered to the reinforcement pre-preg layer that is tacky.

Step D: Air Mesh Casting

An optional air-mesh may be adhered as the outer layer to provide excellent surface finish and attractive colors and may comprise at least one non-adhesive side. The whole assembly of multiple layers is then and wrapped over the scaffold to take its shape.

Step E: Oven Curing

The final multi-layered assembly is cured at elevated temperature, after which the resin will cure to become rigid and the layers will permanently take the shape of the scaffold.

Step F: Removal of Scaffold

Depending on the type material used as the scaffold, the scaffold can be removed upon curing of the article.

### EXAMPLES

Non-limiting examples of the invention and a comparative example will be further described in greater detail by reference to specific Examples, which should not be construed as in any way limiting the scope of the invention.

### Example 1

### Preparation of Pre-Preg Laminate

The reinforcement fibers (glass fiber/carbon fiber/polymeric fiber) are knitted into the form of a sleeve or sock. The reinforcement fibers may or may not be pre-impregnated with the matrix resin. If the reinforcement fibers have been pre-impregnated with resin, the resin should be non-tacky so that they can be knitted easily. The knitting provides stretchability to the fabric since the reinforcement fibers are not stretchable. This stretchability is important to allow the material to easily and snugly conform to shapes without the need of external holding forces, such as the use of molds or vacuum bags.

Reinforcements are impregnated with the matrix resin to form a pre-preg. The pre-preg is then B-staged at temperatures of between 30-90° C. in order to allow the resin to become semi-solid. This step is not necessary if the reinforcement fibers have been pre-impregnated with the matrix and B-staged prior to knitting the fibers into a fabric.

### Example 2

### Preparation of Molded Article

Inner Miura-Ori deployment layer is adhered to the reinforcement pre-preg layer that is tacky. An optional air-mesh can be adhered as the outer layer to provide excellent surface finish and attractive colors. The whole assembly of multiple layers is wrapped over the scaffold to take its shape.

The final multi-layered assembly is cured at elevated temperature, after which the resin will cure to become rigid and the layers will permanently take the shape of the scaffold.

Depending on the type material used as the scaffold, it can be removed upon curing of the article.

### Example 3

### Measurement of Tensile Properties

The tensile properties of the reinforcement layer were determined according to ASTM D3039 standards and compared to a conventional polypropylene (PP) material. The mechanical properties of the reinforcement layer are significantly more superior as compared to the other layers in the laminate, thus the mechanical properties of this reinforcement layer was used as a representative of the laminate. ASTM D3039 determines the in-plane tensile properties of polymer matrix composite materials reinforced with high modulus fibers.

Briefly, a thin flat strip of the material having a constant rectangular cross-section was mounted in the grips of a mechanical testing machine and monotonically loaded in tension while recording load. The ultimate strength of the material was determined from the maximum load carried prior to failure.

The testing was carried out at room temperature. Crosshead speed was controlled at 2 mm/min.

The laminate comprises or consists of the following:

TABLE 1

| Laminate | Properties |
|---|---|
| Air-mesh Layer | Air-mesh provides breathability as well as stretchability. It |
| | enhances functionality to the product and also significantly |
| | reduces manufacturing time. |
| Reinforcement Layer | Provides adequate strength to the product. Knitted fiber loop |
| | structures generates open spaces between the yarns and |
| | facilitates breathability as well as provide stretchability. |
| Inner (Miura-Ori) Layer | Miura-Ori structure allows in-plane airflow through its open |
| | channels. The protruded design on the Miura-Ori facilitates |
| | the interlocking of inner layer to the reinforcement layer. |

The full laminate thickness is about 4-5 mm The reinforcement layer was cut into test samples with dimensions of 175 mm*25 mm*2 mm (L*W*T).

The comparative polypropylene material used in this test is as described below in Comparative Example 1.

The results are summarized in Table 2.

TABLE 2
Mechanical Property Comparison between laminate
of present invention and conventional PP Sheet

| | Modulus | |
|---|---|---|
| Samples | (GPa) | Strength (MPa) |
| Breathable pre-preg | 3.8 | 32 (course |
| Laminate | | direction) |
| PP | 0.88 | 23.6 |

As shown in Table 2, the laminate of the present invention shows superior properties in terms of tensile properties when compared to conventional PP. The present laminate article possesses a high modulus (3.8 GPa) and a high strength (32 MPa) whereas the PP article shows significantly poorer performance at 0.88 GPa and 23.6 MPa. The knitted structure of the reinforcement fabric is characterized by the direction of interlocking loops. The meandering path of the yarn through the fabric is known as the course direction.

### Comparative Example

### Comparative Example 1

### Preparation of PP (Comparative Example)

The polypropylene copolymer manufactured by North Sea Plastic was used to mold sheets with a thickness of 3.1 mm The PP sheet was cut into test samples with a dimension of 175 mm*25 mm*2 mm (L*W*T).

### INDUSTRIAL APPLICABILITY

The disclosed laminate allows for the manufacturing of molded articles with improved mechanical strength. Due to the improved mechanical strength, thin molded articles can be made. The laminate can be molded into any desirable shape. Such molded article may have numerous uses for which can be mentioned the use as a brace for scoliosis, a prosthetic, a sport protector or a safety device.

The disclosed laminate allows for the manufacturing of devices with good ventilation as needed for example in the field of body braces that cover a large body area. The laminates according to the invention may further lead to molded articles wherein all layers are adhered to each other without the need for additional adhesive layer(s). The laminates can be used to make devices which need resistance against wear and tear problems.

The disclosed laminate may also be used in customizable support structures for construction such as molds for concrete, large claddings, temporary structures/barriers, protective housings for equipment, wearable supports/protectors such as genouillere, elbow support, and leg guard, safety helmets for cycling, skate-boarding, customized furniture and structures for bikes and scooters.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1-30. (canceled)

31. A laminate for making a molded article comprising:
(i) at least one reinforcement layer impregnated with a resin matrix;
(ii) at least one deployable layer; and
(iii) optionally, at least one material comprising at least one non-adhesive side.

32. A laminate according to claim 31, wherein the (i) at least one reinforcement layer is a middle layer, the (ii) at least one deployable layer is an inner layer and the (iii) optionally, at least one material comprising at least one non-adhesive side is an external layer.

33. A laminate according to claim 31, wherein the (ii) at least one deployable layer, and the (iii) optionally, at least one material comprising at least one non-adhesive side, are adhered to the surface of the reinforcement layer(s) by the resin matrix, wherein the adhesion is by the cured resin.

34. A laminate according to claim 31, wherein the (iii) optionally, at least one material comprising at least one non-adhesive side is permanently bonded to the reinforcement layer.

35. A laminate according to claim 31, wherein the laminate comprises:
(i) a reinforcement layer impregnated with a resin matrix; and
(ii) a deployable layer; and
(iii) optionally, a material comprising at least one non-adhesive side.

36. A laminate according to claim 31, wherein the (iii) optionally, at least one material comprising at least one non-adhesive side is selected from the group consisting of polyester, nylon, carbon, aramid, and polyolefin or the non-adhesive side has a knitted structure.

37. A laminate according to claim 31, wherein the material of the reinforcement layer is selected from the group consisting of carbon, glass, para-aramid and polymer fibers.

38. A laminate according to claim 31, wherein the resin matrix comprises an epoxy resin,
wherein the epoxy resin is a bis-phenol-A epoxy resin.

39. A laminate according to claim 38, wherein the resin matrix additionally comprises a filler;
wherein the filler is a nanofiller selected from the group consisting of carbon nanotubes, silica, layered silicates, polyhedral oligomeric silsequioxanes and graphene oxide.

40. A laminate according to claim 31, wherein the deployable layer is a deployable polymer layer comprising polyolefin.

41. A laminate according to claim 31, wherein the deployable layer comprises a pattern of folds, thereby allowing the sheet to be collapsed,
wherein the pattern of folds comprises a grid of parallelograms.

42. A laminate according to claim 31, further comprising an anchoring protrusion.

43. A process for making a laminate for making a molded article comprising:
(i) at least one reinforcement layer impregnated with a resin matrix;
(ii) at least one deployable layer; and
(iii) optionally, at least one material comprising at least one non-adhesive side, comprising the following:
providing a deployable layer;
providing a reinforcement layer;
impregnating the reinforcement layer with the resin matrix and partially curing the resin matrix; and
contacting the reinforcement layer with the deployable layer and hilly curing the resin matrix to form the laminate.

44. A process for making a laminate for making a molded article comprising:
(i) at least one reinforcement layer impregnated with a resin matrix;
(ii) at least one deployable layer; and
(iii) optionally, at least one material comprising at least one non-adhesive side, comprising the following:
providing a deployable layer;
pre-impregnating a reinforcement fiber with a resin matrix and partially curing the resin matrix;
weaving the reinforcement fiber to form a reinforcement layer; and
contacting the reinforcement layer with the deployable layer and fully curing the resin matrix to form the laminate.

45. The process of claim 44, wherein the reinforcement fiber is selected from the group consisting of glass fiber, carbon fiber, polymeric fiber and any mixture thereof.

46. The process according to claim 43, wherein the reinforcement layer is contacted with the deployment layer to form a multilayer assembly before curing the resin matrix.

47. The process according to claim 46, comprising wrapping the multi-layer assembly around a scaffold before fully curing the resin matrix or
comprising removing the scaffold after curing the resin matrix.

48. The process according to claim 43, comprising contacting the cured laminate with at least one material comprising at least one non-adhesive side.

49. The process according to claim 43, comprising anchoring the deployment layer to the reinforcement layer;
wherein the anchoring is done using an anchoring protrusion.

50. A molded article obtainable by molding a laminate according to a process for making a laminate for making the molded article comprising:
(i) at least one reinforcement layer impregnated with a resin matrix;
(ii) at least one deployable layer; and
(iii) optionally, at least one material comprising at least one non-adhesive side;
comprising the following:
providing a deployable layer;
providing a reinforcement layer;
impregnating the reinforcement layer with the resin matrix and partially curing the resin matrix; and
contacting the reinforcement layer with the deployable layer and fully curing the resin matrix to form the laminate.

51. A method of using a molded article as a brace for scoliosis, a prosthetic, a sport protector or a safety device, wherein the molded article is obtainable by molding a laminate according to a process for making a laminate for making the molded article comprising:
(i) at least one reinforcement layer impregnated with a resin matrix;
(ii) at least one deployable layer; and
(iii) optionally, at least one material comprising at least one non-adhesive side; comprising the following:
providing a deployable layer;
providing a reinforcement layer;
impregnating the reinforcement layer with the resin matrix and partially curing the resin matrix; and
contacting the reinforcement layer with the deployable layer and fully curing the resin matrix to form the laminate.
